(19) United States
(12) Patent Application Publication
(10) Pub. No.: US 2006/0030888 A1
(43) Pub. Date: Feb. 9, 2006

(54) Static device

(75) Inventor: Gunter Woog, West Bend, WI (US)

Correspondence Address: WEISS & WEISS, Suite 251, 300 Old Country Road, Mineola, NY, 11501, US

(73) Assignee: USI INTERNATIONAL

(21) Appl. No.: 11/122,882

(22) Filed: May 5, 2005

Related U.S. Application Data
(63) Continuation-in-part of application No. 10/912,458, filed on Aug. 5, 2004.

(51) Int. Cl. A61N 1/00

(52) U.S. Cl. 607/2

(57) ABSTRACT

The present invention relates to a method and device for creating a static electric charge and using the device to treat ailments in the body.

### RELATED APPLICATIONS

The present application is a continuation-in-part of pending application Ser. No. 10/912,458 filed Aug. 5, 2004.

### FIELD OF THE INVENTION

The present invention relates to a method and device for creating a static electric charge and using the device to treat ailments in the human body.

### BACKGROUND OF THE INVENTION

There has been a tremendous amount of research in relation between electricity, electromagnetic fields and living organisms. Previous researchers have shown that abnormal physiology is accompanied by abnormal electrical charge. In the normal healing process electrons flow to the abnormal area. There is evidence that artificially interfering with or enhancing the flow of electrons interferes with or enhances the healing process. This process has been used for over two decades to stimulate bone healing. Acupuncture points and meridians are low resistance electrical pathways through which minute D.C. currents flow. Human bodies are continually producing minute D.C. currents through both mechanical and chemical means. These currents follow the paths of least resistance which are in essence like an electric grid for the body. The body in turn uses these currents to regulate a variety of important functions. Acupuncture also alters the flow of current in the body and can thus have very powerful effects apart from any biochemical or neurologic mechanism.

In the past persons have used a tremendous amount of energy to create a static charge in an object. For example, persons have rubbed a mitt vigorously against a PVC pipe to create a static charge.

### SUMMARY OF THE INVENTION

The present invention relates to a device and method for creating a static charge and then using the device to treat an ailment. The device comprises a hollow tube having rough edges or rough texture inside which has a removable piece which creates a static charge on the tube when it is removed. Examples of materials which are removed from the tube which lose electrons are: wool, silk, nylon, glass, fur, hair, lead, aluminum, paper and cotton. Examples of the tube material are: butyrate, wood, amber, rubber, nickel, copper, brass, silver, gold, platinum, polyester, styrene, saran wrap, polyurethane, polyethylene, polypropylene, vinyl, silicon or Teflon.

When an object gains electrons, it becomes negatively charged. An object that is negatively charged repels electrons. By sweeping the negatively charged object near the body, electrons are pushed toward the symptomatic area. This facilitates the normal healing process. In some cases abnormal physiology is caused by an injury and abnormal electrical charge ensues. In other cases an abnormal electrical process can initiate abnormal physiology. In either case using electrostatic massage (EM) will induce physiologic changes through the movement of electrons to the affected area.

EM moves water. Typically areas of injury/inflammation have associated edema and an even higher amount of water than normal. By moving water accumulated in an area of inflammation, pressure on nerves can be reduced and inflammatory byproducts can be dissipated by the present invention.

A positive response to the electrostatic treatment created by the present invention resulted in an improvement in range of motion, decreased tenderness or subjectively decreased pain after treatment. The treatment worked on conditions for muscular pain, sinus headache, arthritis, tension headache, fibromyalgia and rotator cuff tendonitis.

It is an object of the present invention to assist in the relief of aches, pains, stiffness and sore muscles. It is an object of the present invention to provide pain relief within a few minutes. It is an object of the present invention to work in humidity exceeding 50%. It is an object of the present invention to normalize the flow of body electricity.

The present invention relates to a wool activator stored in a plastic tube, wherein in the plastic tube has rough edges or rough texture inside of it, the tube being made of a material which accepts electrons. The wool activator is simply removed from the tube and set aside. The tube has then been charged. The tube is then moved across the effected area approximately ½″ away from the skin. After approximately five passes over the afflicted area, the tube is recharged by reinserting the wool activator in the sleeve and simply removing it. The above process is repeated until the pain subsides, or repeated for up to 15 minutes if necessary.

It is an object of the present invention for the inside of the plastic tube to have a sand paper-like structure.

The present invention relates to a device for creating a static charge used to treat an ailment in the body comprising: a tube having a lining and a rod which fits inside the tube. The rod has a static charge when it is removed from the tube.

It is an object of the present invention for the tube lining to be selected from the group consisting of: wool, silk, nylon, glass, fur, hair, lead, aluminum, paper and cotton.

It is an object of the present invention for the rod to be selected from the group consisting of: butyrate, wood, amber, rubber, nickel, copper, brass, silver, gold, platinum, polyester, styrene, saran wrap, polyurethane, polyethylene, polypropylene, vinyl, silicon and Teflon.

It is an object of the present invention for the device to improve a user's range of motion, decreased tenderness and decreased pain after treatment.

It is an object of the present invention for the device to work in humidity exceeding 50%.

It is an object of the present invention for the tube lining to comprise lambs wool and for the rod to comprise Cellulose Acetate Butyrate.

It is an object of the present invention for the rod to further comprise a handle.

The present invention relates to a method for creating a static charge used to treat an ailment in the body comprising: placing a rod comprising a material that gains electrons inside a hollow tube having a lining made of a material that loses electrons. The rod is removed from the tube, charging the rod. The tube is then set aside. The rod is moved over an effected area approximately ½″ away from the skin. The rod is passed over the effected area until the rod no longer has a charge. The rod is recharged by reinserting the rod in the tube and removing it. The process is repeated until the pain subsides, or repeated for up to 15 minutes if necessary.

The invention will be further set forth in the detailed description, accompanying claims and in the drawings.

### BRIEF DESCRIPTION OF THE DRAWINGS

FIG. 1 is a view of the device in the closed position;

FIG. 2 is a view of the device in the open position where the wand is removed from the tube;

FIG. 3 is a view of the device in the closed position where the rod is inserted into the tube;

FIG. 4 shows the rod separate from the tube;

FIG. 5 shows the tube separate from the rod;

FIG. 6 is a top view of the tube;

FIG. 7 is a cut away view of the tube;

FIG. 8 is a view of the device in the closed position;

FIG. 9 is a view of the device in the open position where the wand is removed from the tube.

### DETAILED DESCRIPTION OF THE INVENTION

The present invention relates to a device and method for creating a static charge and then using the device to treat an ailment. The device comprises a hollow tube having rough edges or a rough texture inside the tube. The device has a removable piece which creates a static charge on the tube when it is removed. Examples of materials which are removed from the tube which lose electrons are: wool, silk, nylon, glass, fur, hair, lead, aluminum, paper and cotton. Examples of the tube material are: butyrate, wood, amber, rubber, nickel, copper, brass, silver, gold, platinum, polyester, styrene, saran wrap, polyurethane, polyethylene, polypropylene, vinyl, silicon or Teflon.

In a preferred embodiment lambs wool is used for the part of the device that gives up the charge, and the tube is made of clear or colored Cellulose Acetate Butyrate.

The device of the present invention was used with a number of persons who complained about aches and pains in different regions of their body. A positive response to the electrostatic treatment resulted in an improvement in range of motion, decreased tenderness or subjectively decreased pain after treatment. The treatment worked on conditions for muscular pain, sinus headache, arthritis, tension headache, fibromyalgia and rotator cuff tendonitis. The device even worked in conditions of humidity exceeding 50%.

The device of the present invention assists in the relief of aches, pains, stiffness and sore muscles, and provides the pain relief within a few minutes. The device of the present invention normalizes the flow of body electricity.

The present invention places a wand inside a hollow tube that has rough edges inside the tube. The rough edges inside the tube assist in generating a greater static electric charge. The wand is stored in the hollow tube until it is needed. The wand which is made of material that loses electrons is simply removed from the tube and set aside. The tube has then been charged. The tube is then moved over the effected area approximately ½″ away from the skin. After approximately five passes over the afflicted area, the tube is recharged by reinserting the wand activator in the sleeve and simply removing it. The above process is repeated until the pain subsides, or repeated for up to 15 minutes if necessary.

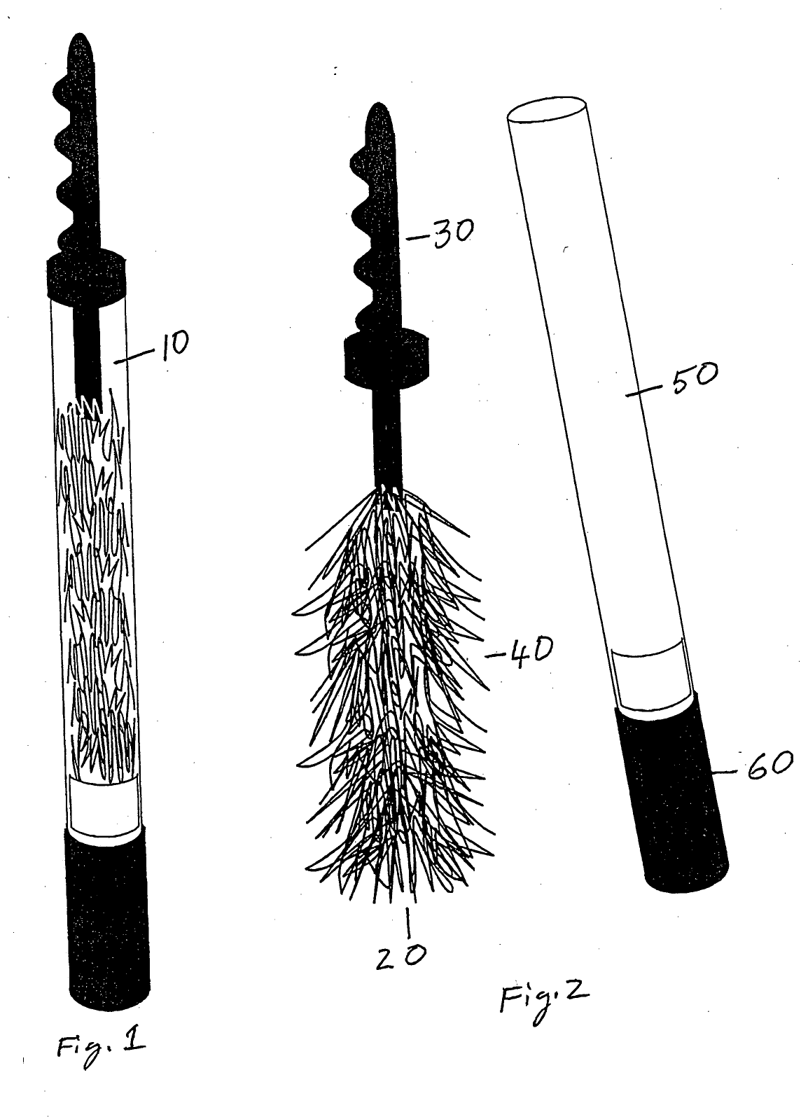

FIG. 1 illustrates the device of the present invention in the closed position. FIG. 2 illustrates the device of the present invention in the open position. Device 10 comprises a wand 20 made of a handle 30 and a material which gives up electrons 40. FIGS. 1 and 2 show 40 as lamb wool. The wand 20 is inserted into tube 50, which is made of material that accepts electrons. FIGS. 1 and 2 show tube 50 as clear or colored Cellulose Acetate Butyrate. When the wand 20 is removed from the tube 50 while held by handle 60, tube 50 is given an electrical charge. The tube 50, held by handle 60 is then placed over the affected area and passed over the area about 5 times until there is no charge left on the tube. The tube 50 can then be repeatedly recharged by placing the wand 20 back inside the tube 50 and removing it again.

FIG. 3 shows a device 100 having a rod 110 inserted into a tube 120. The tube 120 has a lambs wool lined sheath 130.

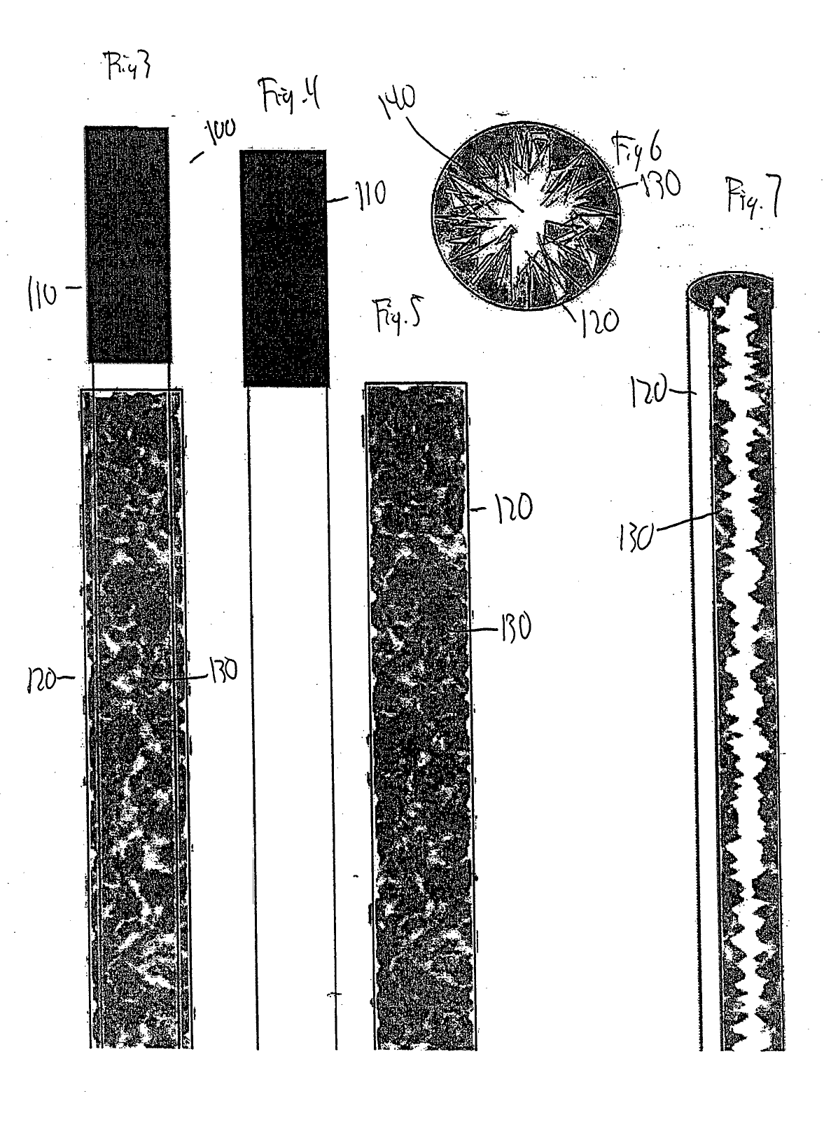

FIG. 4 shows the rod 110 separate from the tube 120.

FIG. 5 shows the tube 120 by itself without the rod 110. The tube 120 has a lamb wool lined sheath 130 inside it.

FIG. 6 shows a top view of the tube 120 being lined with a lamb wool sheath 130 having an opening 140 to place the rod 110.

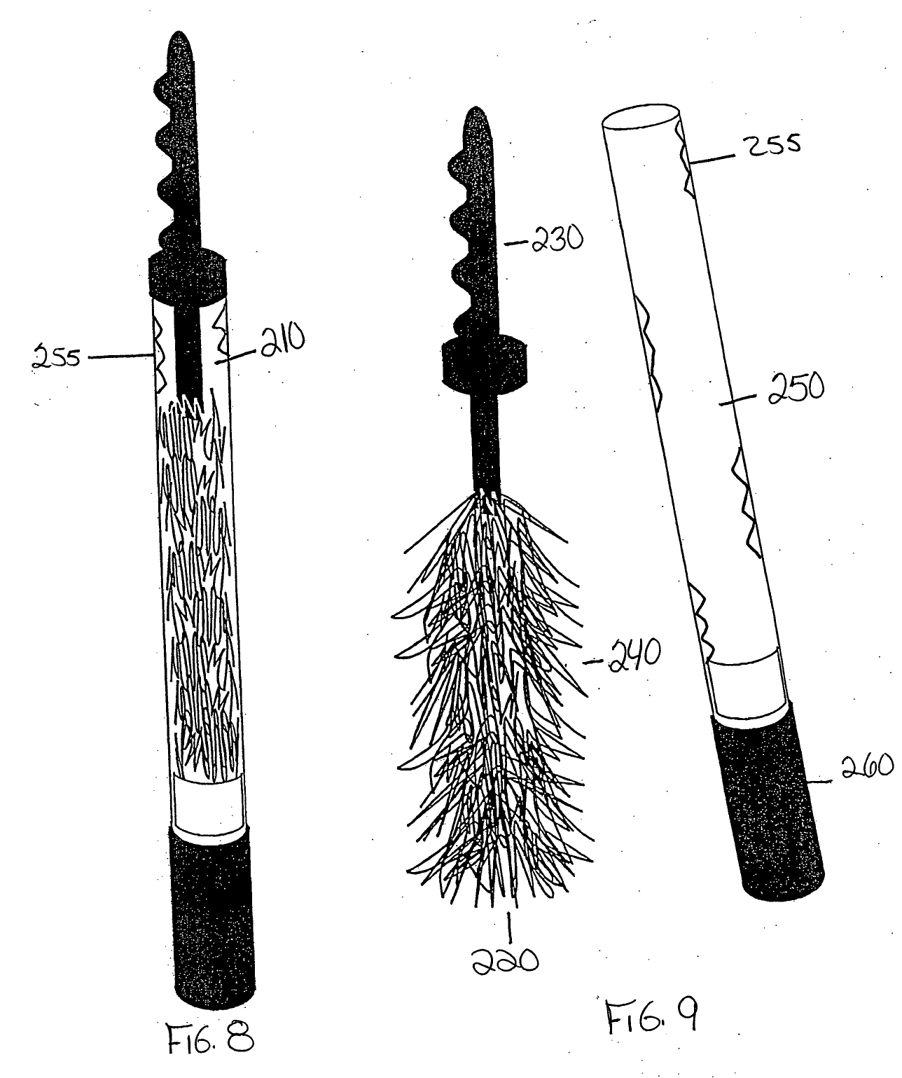

FIG. 7 shows the tube 120 having the lamb wool lined sheath 130.

FIG. 8 illustrates the device of the present invention in the closed position. FIG. 9 illustrates the device of the present invention in the open position. Device 210 comprises a wand 220 made of a handle 230 and a material which gives up electrons 240. FIGS. 8 and 9 show 240 as lamb wool. The wand 220 is inserted into tube 250 having rough edges or a rough texture 255, which is made of material that accepts electrons. FIGS. 8 and 9 show tube 250 as clear or colored Cellulose Acetate Butyrate. When the wand 220 is removed from the tube 250 while held by handle 260, tube 250 is given an electrical charge. The tube 250, held by handle 260 is then placed over the affected area and passed over the Area about 5 times until there is no charge left on the tube. The tube 250 can then be repeatedly recharged by placing the wand 220 back inside the tube 250 and removing it again.

It will be apparent to those skilled in the art that various modifications of the foregoing illustrative embodiment are possible. Thus, the invention also encompasses any and all embodiments within the scope of the following claims.

1. A device for creating a static charge used to treat an ailment in the body comprising:
a hollow tube having rough edges or a rough texture inside said tube;
a wand which fits inside said hollow tube;
said wand creating a static charge on said tube when it is removed from said tube.

2. The device of claim 1 wherein said wand material is selected from the group consisting of: wool, silk, nylon, glass, fur, hair, lead, aluminum, paper and cotton.

3. The device of claim 1 wherein said tube material is selected from the group consisting of: butyrate, wood, amber, rubber, nickel, copper, brass, silver, gold, platinum, polyester, styrene, saran wrap, polyurethane, polyethylene, polypropylene, vinyl, silicon and Teflon.

4. The device of claim 1 wherein said device results in an improvement in range of motion, decreased tenderness and decreased pain after treatment.

5. The device of claim 1 wherein said device works in humidity exceeding 50%.

6. The device of claim 1 wherein said wand comprises lambs wool and said tube comprises Cellulose Acetate Butyrate.

7. The device of claim 1 wherein said wand further comprises a handle.

8. A method for creating a static charge used to treat an ailment in the body comprising:
placing a wand comprising a material that loses electrons inside a hollow tube made of a material that gains electrons;
said hollow tube having rough edges or a rough texture inside said tube;
removing said wand from said tube, charging said tube and setting said wand aside;
moving said tube over an effected area approximately ½″ away from the skin;
passing said tube over said effected area until said tube no longer has a charge;
recharging said tube by reinserting said wand in said tube and removing it; and
repeating said process until pain subsides.
